(19) United States
(12) Patent Application Publication
(10) Pub. No.: US 2024/0261023 A1
(43) Pub. Date: Aug. 8, 2024

(54) DEVICE WITH MULTI-EMITTERS OF LASER ENERGY AND ASSOCIATED ASSEMBLY FOR PERFORMING HEAT TREATMENT

(71) Applicants: Universite de Bordeaux, Bordeaux (FR); INSERM (Institut National de la Santé et de la Recherche Medicale), Paris (FR); Fondation Bordeaux Universite, Bourdeaux (FR)

(72) Inventor: Bruno Quesson, Cestas (FR)

(21) Appl. No.: 18/563,823

(22) PCT Filed: Mar. 25, 2022
(86) PCT No.: PCT/FR2022/050564

§ 371 Date: Nov. 22, 2023

(30) Foreign Application Priority Data
May 25, 2021 (FR) FR2105430

(51) Int. Cl. A61B 18/22; A61B 5/01; A61B 5/055; A61B 18/00; A61B 18/20; A61N 5/00; A61N 5/06; A61N 5/067; G01K 11/32

(52) U.S. Cl. CPC A61B 18/22; A61B 5/015; A61B 5/055; A61N 5/062; A61N 5/067; G01K 11/32; A61B 2018/00023; A61B 2018/00351; A61B 2018/00702; A61B 2018/00714; A61B 2018/00791; A61B 2018/2065; A61B 2018/2211; A61N 2005/005; A61N 2005/0626; A61N 2005/063

(57) ABSTRACT

A laser device for treating a target region of biological tissue comprises: a sheath; at least two optical fibers in the sheath, each configured to guide a laser beam to the target region, distal ends of at least two of the optical fibers configured so that each emits a laser beam in a different direction; a system of laser sources configured to generate at least two laser beams, the two laser beams having different or identical wavelengths with adjustable power; and a laser-beam control unit configured to control the system of laser sources so as to select the wavelength, the power, the duration of the deposition of laser energy, and the moment of emission of each of the laser beams in the direction of the target region so as to dynamically generate and adjust a 3D thermal distribution having a geometric shape matching a geometric shape of the target region.

### CROSS-REFERENCE TO RELATED APPLICATIONS

This application is a national phase entry under 35 U.S.C. § 371 of International Patent Application PCT/FR2022/050564, filed Mar. 25, 2022, designating the United States of America and published as International Patent Publication WO/2022/248778 A1 on Dec. 1, 2022, which claims the benefit under Article 8 of the Patent Cooperation Treaty of French Patent Application Serial No. FR2105430, filed May 25, 2021.

### TECHNICAL FIELD

The present disclosure relates to the field of treating a biological tissue using localized variation in temperature under guidance by intraoperative imaging.

More specifically, the present disclosure relates to a device with multi-emitters of laser energy capable of emitting a plurality of laser beams to induce in a biological tissue a 3D temperature variation corresponding to a predefined target region of any shape, and potentially asymmetric.

The present disclosure also relates to a heat treatment assembly comprising such a device with multi-emitters of laser energy coupled to an MRI imaging device.

### BACKGROUND

It is known to treat pathological biological tissues locally by targeted administration of an increase in temperature (hyperthermia) or a decrease in temperature (hypothermia) by means of an energy source. For example, the energy may be provided by a laser, microwaves, radio-frequency waves, focused ultrasound or by cryo-therapy.

Among these techniques, a first category of heat treatment is distinguished that consists in depositing an energy dose in a target region of a biological tissue via energy generator means positioned remotely (focused ultrasounds or radio-frequency waves by induction) and a second heat treatment category that consists of depositing a dose of energy in the target region by percutaneous or vascular route (radio-frequency, laser, microwave, cryotherapy). The heat treatment system of the present disclosure belongs to the second category.

Prior to the heat treatment, a phase referred to as “pre-operative planning phase” is intended to assess the 3D extension of the target region thanks to suitable imaging techniques, for example, by computed tomography (which may be designated by “TDM”) or by Magnetic Resonance Imaging (which may be designated by “MRI”), able to determine the size, the number, the location and the shape of the target region(s).

During this pre-operative planning phase, global indicators of the dimensions of the target region, their number and their relative location with respect to identifiable anatomical references are generally defined.

The planning phase also aims to prepare the treatment, which involves defining the treatment instructions, namely the dose of thermal energy to be delivered in a volume as a function of the functional characteristics of the biological tissue to be treated, of the size of the target region, and of the severity of the pathological tissue.

For a treatment to be effective, a target region is defined that comprises the pathological tissue visible in imaging and optionally a minimum safety margin to be observed, defined by the practitioner around the pathological tissue. This target region should undergo a temperature variation adapted to treat the pathological tissue.

The target region is generally surrounded by a region whose tissue is healthy and should ideally not undergo deleterious thermal variation during the heat treatment. In this region surrounding the target region, one or more critical regions to be preserved (vital organs and/or structures) can be distinguished.

In the region where the tissue is healthy and does not include critical regions, the tissues should ideally not undergo temperature variation during the heat treatment. Nonetheless, a possible temperature variation is not considered to be critical for the patient.

Although the heat treatment technique is much less invasive than surgery, it has some drawbacks.

One of the main limitations of the efficiency of this technique is due to the arbitrary shape of the target region to be treated. Indeed, in known hyperthermal treatment devices, the energy deposited generally aims to heat a spherical or ellipsoidal volume around the application point. However, the proposed devices do not make it possible to adjust the shape of the lesion generated by the applicator to the shape of the target region to be treated. On the other hand, the distribution of heat in the tissues depends on their intrinsic thermal characteristics (absorption, thermal diffusivity, perfusion) and often leads to a modification of the spatial distribution of the temperature relative to the thermal distribution planned by the practitioner. Therefore, the energy deposited does not make it possible to guarantee a complete treatment of all the target regions.

The lack of adaptability between the shape of the effective temperature distribution and the shape of the target region can lead to insufficient energy deposition in certain areas of the target region and/or any undesired energy deposition in a critical region to be preserved. One of the consequences of this lack of adaptability of the shape of the lesion is the increase in the number of incomplete treatments and the associated risks of local recurrence. Likewise, the risks of altering healthy biological tissues are accentuated, increasing the risks of potentially serious side effects.

It is known to use an optical fiber or a set of optical fibers to deposit a laser energy dose in contact with the target region. Indeed, the use of optical fibers makes it possible to bring its distal end into direct contact with the target region and to deposit therein the thermal energy required by absorbing light energy emitted using a laser source.

A known example embodiment is a device that comprises a main sheath integrating an optical fiber or a set of optical fibers. The distal end of the sheath comprises an opening through which the end of the optical fiber or of the set of optical fibers emits an irradiation light energy intended to locally treat the target area. This solution makes it possible to bring the optical fiber as close as possible to the target zone. However, it does not make it possible to meet all the technical constraints indicated above.

One of the aims of the present disclosure is therefore to provide an emission device having a plurality of optical fibers whose emission direction can be different for each fiber and dynamically adjustable during the treatment, in order to be able to create a treatment in accordance with the therapeutic objective.

Another aim of the present disclosure is to be able to propose a device that is capable of controlling and modulating the light power and the wavelength of each laser fiber emitted independently. The adjustment of the wavelength makes it possible to modulate the induced warming depth, since the biological tissues absorb light differently as a function of their wavelengths.

Another aim of the present disclosure is to be able to propose a device that is capable of controlling and modulating the light power and the moment of emission of each optical fiber in order to generate and adjust in real time during the treatment a 3D distribution with a geometric shape adapted to the geometric shape of the target region.

Another aim of the present disclosure is to provide a device for measuring in real time the temperature at the distal end of the device, thus providing a means for measuring temperature in addition to the thermometric imaging system.

Other aims and advantages of the present disclosure will become apparent from the following description, which is however only given by way of indication and is not intended to limit it.

### BRIEF SUMMARY

A laser device with multi-emitters of laser energy is proposed for heat-treating a target region of a biological tissue, comprising:
at least one sheath having a longitudinal axis (AA′) and comprising a proximal end and a distal end that is intended to be placed facing the target region;
at least two optical fibers extending, in the sheath, between the proximal end and the distal end, each of the optical fibers being suitable for guiding a heat-treatment laser beam to the target region and depositing laser energy in the target region;
wherein the distal ends of at least two optical fibers are configured so that each emits a laser beam in a different emission direction with respect to the longitudinal axis of the sheath;
a system of laser sources that is configured to generate at least two laser beams, the at least two laser beams having different or identical wavelengths with an adjustable light power;
a laser-beam control unit configured to control the system of laser sources so as to select the wavelength, the light power, the duration of the deposition of laser energy and the moment of emission of each of the laser beams guided and emitted by the optical fibers in the direction of the target region so as to generate and adjust dynamically a 3D thermal distribution having a geometric shape matching the geometric shape of the target region.

The features disclosed in the next paragraphs may optionally be implemented. They may be implemented independently of one another or in combination with one another:

The distal end of at least two optical fibers is positioned at a different distance from a surface of the distal end of the sheath.

The distal end of the optical fibers is configured to emit a laser beam in an emission direction oriented at an angle α between 0 and 180° with respect to the longitudinal axis of the sheath.

The system of laser sources comprises a plurality of monochromatic laser sources.

The system of laser sources is adapted to generate at least two laser beams of different laser wavelengths for each optical fiber.

According to one embodiment of the present disclosure, the device further comprises a plurality of optical transmission fibers able to transmit the laser beams generated by the system of laser sources to the optical fibers of the sheath.

Preferably, the device further comprises a temperature sensor.

According to one embodiment, the temperature sensor is a detection optical fiber capable of detecting a temperature variation in the target region.

According to one embodiment, the plurality of heat-treatment optical fibers is distributed according to a radial symmetry around the detection optical fiber.

Advantageously, the device further comprises connection means capable of connecting the optical fibers of the sheath with the optical transmission fibers of the system of laser sources.

According to a particularly advantageous embodiment, the sheath comprises at least one lumen adapted to inject a therapeutic substance under pressure intended to be ejected toward the target region.

According to another embodiment, the sheath comprises a closed cooling circuit suitable for transporting a cooling liquid intended to cool a part of the distal end of the sheath.

According to one variant, the closed cooling circuit is formed by at least two openings provided in the sheath.

Preferably, the cooling circuit is formed by a cooling sheath surrounding the sheath comprising the optical fibers and a lumen provided in the sheath.

According to another aspect, a heat treatment assembly is provided for a target region of a biological tissue comprising:
a laser device with multi-emitters of laser energy as defined above for treating the target region; and
a magnetic resonance imaging system configured to generate anatomical images and thermometric images of the target region.

### BRIEF DESCRIPTION OF THE DRAWINGS

Other features, details and advantages will appear upon reading the detailed description hereinafter, and upon analyzing the appended drawings, wherein:

FIG. 1 shows a laser device with multi-emitters of laser energy according to one embodiment of the present disclosure;

FIG. 2 shows a laser device with multi-emitters of laser energy according to another embodiment of the present disclosure;

FIG. 3 schematically shows a perspective view of a sheath comprising an assembly of optical fibers;

FIG. 4 schematically shows a side and sectional view of a set of three treatment optical fibers in a sheath according to one embodiment;

FIG. 5 schematically shows a perspective view of an embodiment wherein the device comprises three sheaths, each of the sheaths comprising an assembly of five optical fibers;

FIG. 6A shows a cross-sectional front view of an embodiment of a sheath provided with five lumens intended to receive treatment optical fibers and a sixth central lumen intended to receive a temperature sensor or to inject a cooling liquid or a therapeutic substance;

FIG. 6B shows a cross-sectional front view of an embodiment of a sheath of FIG. 6A provided with two additional lumens to form a closed cooling circuit;

FIG. 6C shows a cross-sectional front view of an example embodiment of an optical sheath of FIG. 6A surrounded by a cooling sheath to form a closed cooling circuit with the central lumen;

FIG. 7 shows a heat treatment assembly according to one embodiment of the present disclosure comprising a laser device with multi-emitters coupled to an MRI imaging device;

FIG. 8A schematically shows a sheath comprising six treatment optical fibers capable of emitting six light energies.

FIG. 8B shows six temperature images obtained by MRI thermometry with the sheath of FIG. 8A, each temperature image being obtained simultaneously with the activation of a single optical fiber and each optical fiber being sequentially activated one after the other;

FIG. 8C shows a temperature image obtained with the sheath of FIG. 8A, during the simultaneous activation of the six optical fibers with the same power;

FIG. 9 schematically shows an example of use with two sheaths whose distal ends are positioned on either side of a target region;

FIG. 10 schematically shows an embodiment of a system of laser sources that can generate two laser beams of different wavelengths for each treatment optical fiber;

FIG. 11 schematically shows a front view of a system of laser sources comprising six optical fibers;

FIG. 12 shows photographs of the individual laser beams emitted by each of the six optical fibers of FIG. 11;

FIG. 13 shows temperature images obtained by MRI thermometry during sequential activation of each of the optical fibers and the curves of the graph show the change of the temperature in six pixels, each pixel being selected in an angular sector covered by a different optical fiber; and

FIG. 14 shows temperature images obtained by MRI thermometry for three different activation configurations generating three geometric thermal distribution shapes: triangle (a), ellipse (b), and semi-circle (c), each image displayed being chosen at the time of the end of the laser emission, corresponding to the maximum increase of the temperature by the selected diodes, the points numbered indicated on each image are the pixels selected to display the curves of temperature over time on the graphs at right.

### DETAILED DESCRIPTION

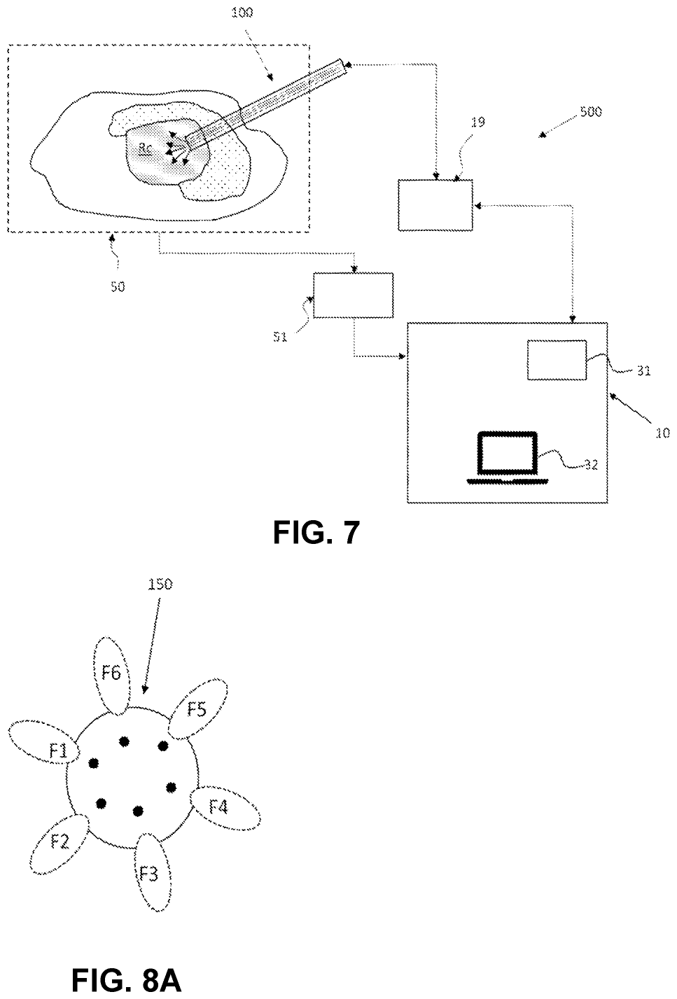

In the context of the present disclosure, by “target region,” it should be understood a region comprising the pathological tissue to be treated visible in imaging and a region that surrounds the pathological tissue. The extent of the neighborhood around the pathological tissue is defined by the practitioner. The target region should undergo a temperature variation in order to treat the pathological tissue. The region is designated Rc in FIG. 7.

In the context of the present disclosure, a 3D anatomical image is a reconstructed image representing the anatomy of the target region and its environment. This 3D anatomical image may be obtained by different imaging techniques.

In the context of the present disclosure, a 3D temperature image is a 3D image representative of a spatial distribution of the temperature of the target region and of the region surrounding it. The 3D temperature image is obtained by an MRI magnetic resonance imaging device, using a temperature sensitive imaging sequence and a real-time image processing device that calculates and displays the temperature variations in the target region and the region surrounding it.

In the context of the present disclosure, “proximal” refers to a piece or part of the device that is located near the operator or the practitioner when they are using the device, while “distal” means a piece or part of the device that is remote from the operator during this use.

For the most part, the drawings and the description hereinafter contain certain elements. Hence, they could not only be used to better understand this disclosure, but also contribute to the definition thereof, where applicable.

In the following, the present disclosure will more particularly be described in the case of a heat treatment of a target region and a detection of temperature variation during the heat treatment. However, this is not limiting, insofar as the system can be used together with the injection of a treatment solution or other types of fluids, introduced into a lumen of the sheath provided for this purpose.

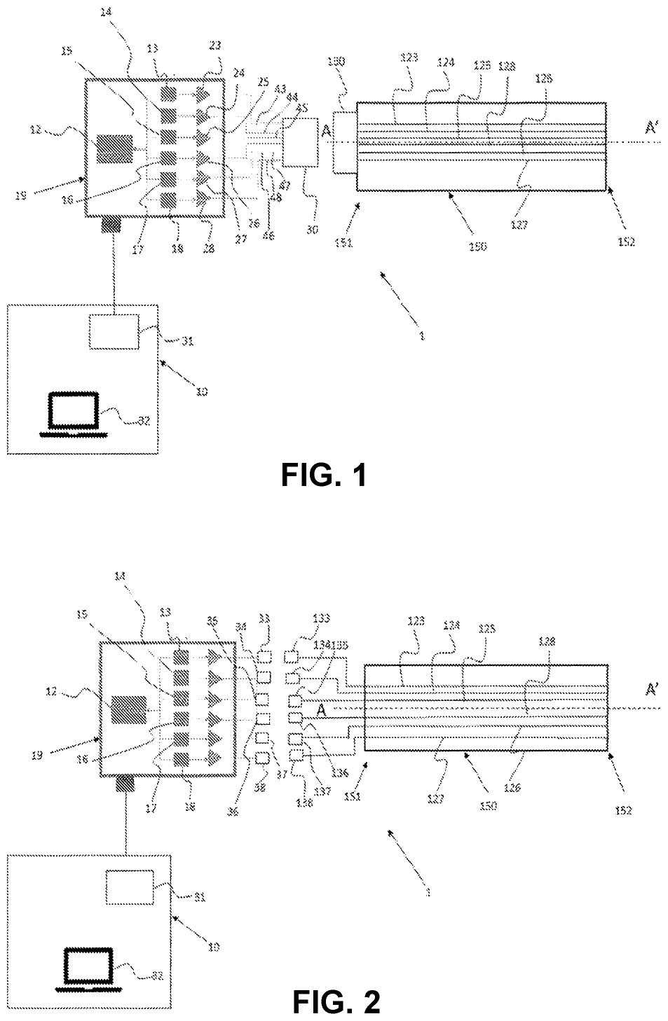

FIG. 1 schematically shows a device 1 with multi-emitters of laser energy according to one embodiment of the present disclosure.

The device 1 comprises a plurality of optical fibers 123, 124, 125, 126, 127, 128 intended to transport a plurality of laser beams toward a target region of a biological tissue, a system of laser sources 19 and a main control unit 10. The system of laser sources 19 is configured to generate a plurality of laser beams intended to be injected into the optical fibers and guided by the optical fibers to the target region. Part of the plurality of laser beams is intended to irradiate the target region so as to induce a variation in temperature and/or to activate molecules present in a solution previously deposited in the target region. The main control unit 10 is configured to control the system of laser sources 19 so as to select the wavelength, the light power, the duration of the laser energy deposition and the emission moment of each of the treatment laser beams for each of the optical fibers.

The device also comprises one or more temperature sensors having the function of measuring the temperature of the target region in contact with the sheath.

According to one embodiment, the temperature sensor is formed by one of the optical fibers and at least one laser beam among the plurality of laser beams emitted by the optical fibers is intended to detect a variation in the temperature of the target region during the heat treatment. The control unit 10 of the laser beams is also configured to receive a detection laser beam coming from this optical fiber dedicated to the measurement of the temperature.

According to one variant, the temperature sensor may be, for example, a thermocouple inserted into one of the lumens of the sheath. The thermocouple is connected to the control unit 10 of the laser beams.

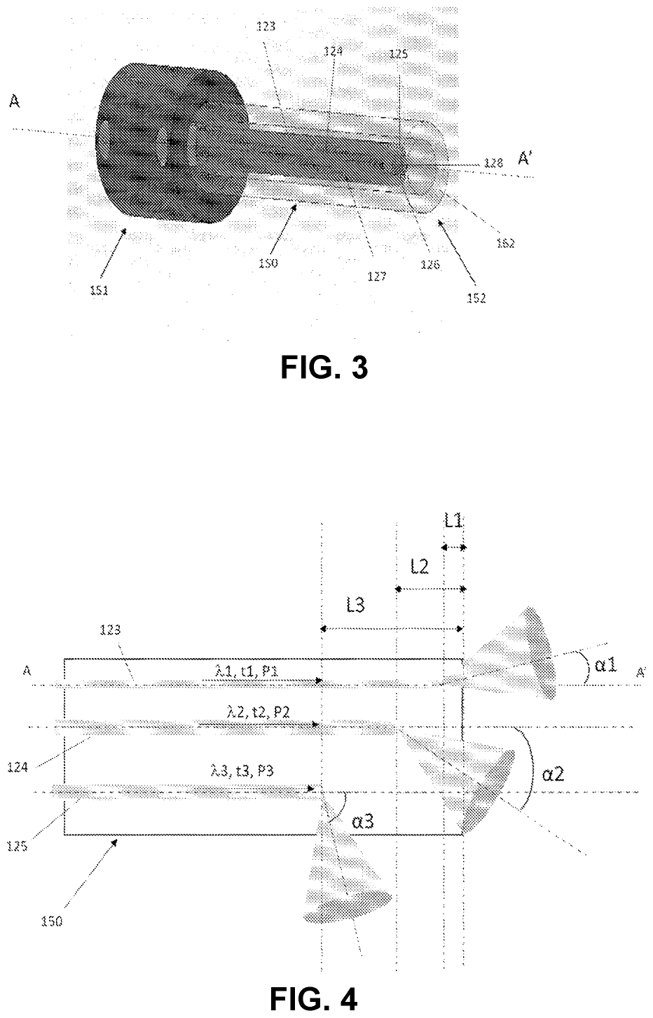

With reference to FIG. 3, the optical fibers 123, 124, 125, 126, 127, 128 are held in a sheath 150 that serves to hold the optical fibers together. The sheath 150 is in the form of a flexible or rigid body, depending on the targeted therapeutic application, of substantially cylindrical shape having a longitudinal axis AA′. The sheath comprises a proximal end 151 and a distal end 152 intended to be placed facing the target region. The sheath 150 is made of a material compatible with a surgical procedure and adapted to allow the light beams emitted by the optical fibers to pass through. The sheath is provided with lumens and each of the lumens contains an optical fiber that extends between the distal end 152 and the proximal end 151 of the sheath 150.

According to a particularly advantageous form, the sheath is in the form of an end piece capable of being detachably connected to the system of laser sources 19. The sheath has, for example, an external diameter of 1.8 mm and an internal diameter of 1.2 mm. The sheath can be covered with a protective surface that can become blackened due to the absorption of the light ray, it will be possible to change the protective surface without changing the set of optical fibers.

According to one embodiment, the optical treatment and detection fibers have a diameter of between 50 μm and 1000 μm, preferably between 100 and 400 microns.

In FIGS. 1, 2 and 3, the five optical fibers 123, 124, 125, 126, 127 are, for example, treatment optical fibers adapted to each transmit a light beam having a wavelength adapted to treat the target region, and the sixth optical fiber 128 is a detection fiber and has the function of measuring a temperature variation of the target region. For the remainder of the description, the terms “treatment fiber” or “emitter” are used to refer to the optical fibers intended to transmit a light beam dedicated to the heat treatment, and the term “detection fiber” is used to refer to the optical fiber dedicated to the detection of a temperature variation.

The five light beams emitted by the five emitters or treatment fibers can each cover, for example, an angular sector with an angle of 72°, so as to emit according to one complete revolution of 360°. According to another variant, the sheath 150 may comprise ten optical emitters that each make it possible to cover an angular sector of 36°. The number of emitters is not limiting. The example of arrangement shown in FIGS. 1, 2, 3 and 4 is not limiting and can vary depending on requirements.

According to one embodiment, the distal end of each of the treatment fibers can be positioned at different distances L relative to the distal end of the sheath, making it possible to modulate the relative position of each of the optical fibers in the length direction of the sheath.

According to one embodiment, the distal end of each of the emitters is configured so as to emit a light beam oriented in a different direction. The distal end of each of the optical fibers is polished, for example, so as to emit a laser beam whose emission direction is oriented at an angle α defined relative to the main axis AA′ of the optical fiber. This angle may be between 0° and 180°. It is thus possible to obtain a set of optical fibers capable of emitting a set of light beams that each illuminate a predefined angular sector.

The combination of the different light beams emitted by the distal end of each of the emitters with a different emission direction and at different distal positions along the sheath makes it possible to generate lesions of dimensions and geometries adapted to the shape of the target region.

FIG. 4 shows an example of a sheath 150 comprising three treatment fibers or emitters 123, 124, 125. The distal end of each of the treatment emitters 123, 124, 125 is positioned, respectively, at a different distance L1, L2, L3 from the distal end 152 of the sheath. Each of the emitters emits a light beam in a different direction, thus covering a different angular sector. Each of the three directions is defined herein by a different angle α1, α2, α3 comprised between the axis of symmetry of the beam and the main axis AA′ of the optical fiber. The angle α may be between 0° and 180°.

According to another embodiment, the device may also comprise a plurality of optical sheaths.

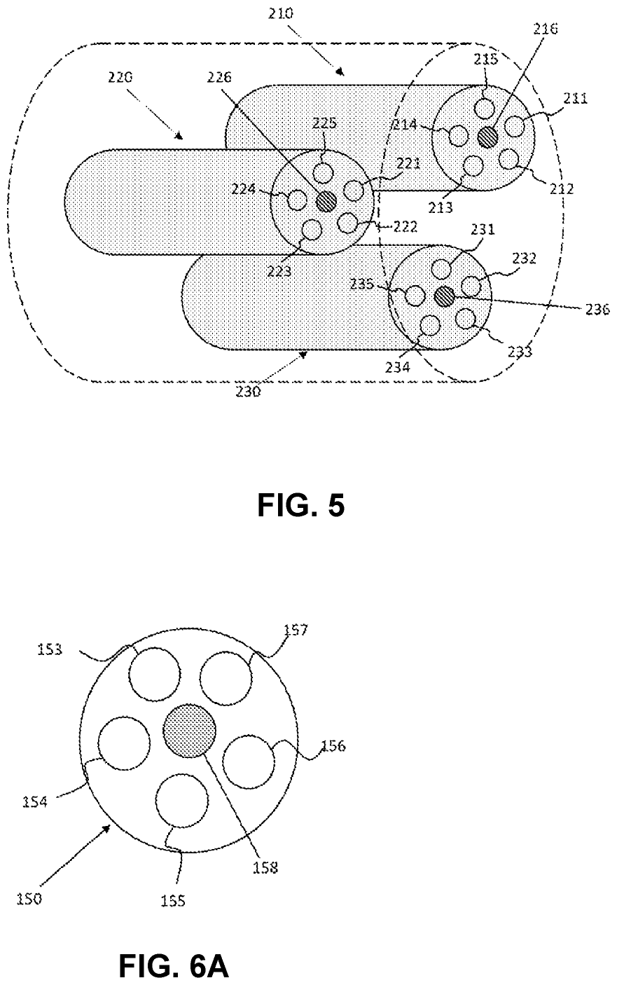

With reference to FIG. 5, the device may comprise, for example, three optical sheaths 210, 220, 230. Each of the optical sheaths 210, 220, 230 here comprises, respectively, five treatment optical fibers 211, 212, 213, 214, 215, 221, 222, 223, 224, 225, 231, 232, 233, 234, 235 and a central detection optical fiber 216, 226, 236. The light beams transported by the fibers of the same set can, for example, have different wavelengths, different powers, different emission times and different emission moments. Depending on the shape of the distal end of the optical fibers and their distal positions relative to the distal end of the sheath, they may have different emission directions and different distal emission positions along the sheath.

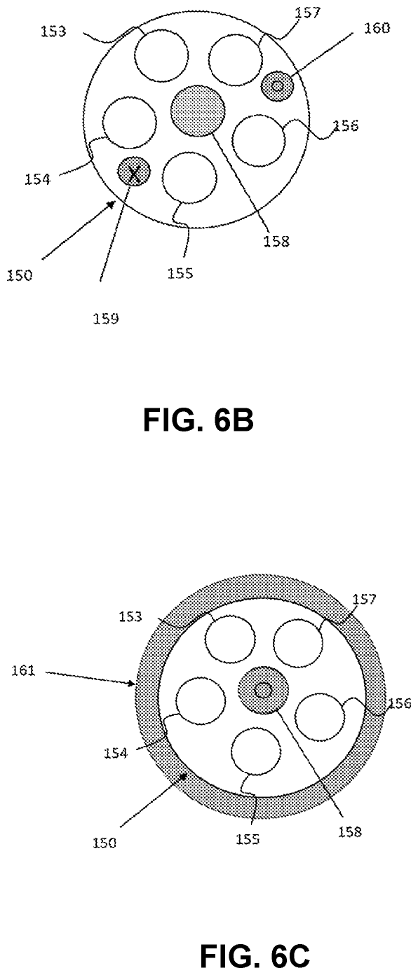

According to one embodiment of the present disclosure and with reference to FIG. 6A, the sheath comprises five lumens 153, 154, 155, 156, 157 arranged along a radial symmetry around a central lumen 158. The peripheral lumens 153, 154, 155, 156, 157 are, for example, each intended to receive a treatment optical fiber. The central lumen 158 of the sheath is intended to pass the temperature sensor, for example, a detection optical fiber 128 or a thermocouple having the function of measuring the temperature. This temperature measurement makes it possible to control any deviation between the temperature measured at the end of the optical fiber and the temperature measured by an MRI Magnetic Resonance Imaging device. A known technique consists of using an optical fiber provided with a Bragg grating inscribed within the core of the fiber. The Bragg grating consists of a periodic and longitudinal modulation of the refractive index of the core of the single-mode fiber. The Bragg grating reflects light at the Bragg wavelength. When the optical fiber is subjected to a temperature variation, the fiber undergoes relative elongation as well as a variation in the refractive index, which results in a variation in the reflected wavelength. Thus, it is possible to measure the temperature by measuring the variation of the wavelength of the reflected light.

According to another embodiment of the present disclosure, the sheath comprises an additional lumen that makes it possible to convey, for example, a therapeutic solution intended to be deposited in the target region. The solution injected is a solution comprising, for example, temperature-activatable molecules, for example, anti-cancer agents encapsulated in a heat-sensitive nanovehicle. According to this embodiment, when the solution is deposited, the emitters or the treatment fibers each emit a light beam toward the target region in order to thermally activate the molecules of the solution.

According to yet another embodiment and with reference to FIG. 6B, the sheath 150 may comprise two lumens 159, 160 intended for the circulation of a cooling liquid by forming a closed circuit and another lumen 158 intended to convey a therapeutic substance. The lumen 160 is intended for the arrival of cooling fluid and the lumen 159 for the return of the cooling fluid.

According to one variant, the central lumen 158 can be used as the inlet of cooling fluid and the other two lumens 159, 160 as return for cooling fluid. All three lumens form a closed circuit.

According to yet another embodiment and with reference to FIG. 6C, the optical sheath 150 that comprises the set of optical fibers is surrounded by a cooling sheath 161. The cooling fluid arrives via the central lumen 158 and is returned via the cooling sheath 161. This configuration of the cooling circuit makes it possible to have more homogeneous cooling over the entire distal part of the sheath.

In the context of an injection of therapeutic substance, the lumen comprises an inlet orifice located on a proximal end surface of the sheath and an outlet or injection orifice on the surface of the distal end of the sheath. The inlet port is connected to a piston intended to inject the therapeutic substance into the lumen provided for this purpose. The flow rate of injection of the solution circulating in the lumen is controlled so that the therapeutic substance can be directed and ejected toward the target region. Other embodiments can be envisaged in order to eject the substance into the target region.

With reference to FIGS. 1 and 2, the system of laser sources 19 and the main control unit 10 are described below.

The system of laser sources 19 is adapted to generate a plurality of laser beams for the heat treatment of the target region and optionally the detection of a temperature variation in the case where the temperature sensor is an optical fiber.

The number of laser beams generated by the system of laser sources 19 is not limiting. According to one embodiment and with reference to FIG. 10, the system of laser sources 19 can generate, for example, two laser beams of different wavelength by treatment optical fiber 123, 124, 125, 126, 127. Thus, for the device of FIG. 1 that comprises five treatment optical fibers and a detection optical fiber 128, the system of laser sources is configured to generate ten treatment laser beams and one detection laser beam.

Preferably, the laser beams generated for each optical fiber can have identical or different wavelengths and light powers. In the example embodiment where the system of laser sources 19 generates two laser beams for each treatment optical fiber, it is therefore possible to select one of the two wavelengths 21 or 22 and one of the two light powers of the treatment light beam transported and emitted by the treatment optical fiber.

According to one embodiment, the laser beams are generated by means of a plurality of monochromatic laser sources. Each of the monochromatic laser sources generates a light beam at a given wavelength. The use of a plurality of treatment wavelengths makes it possible to adjust the penetration depth of the beam into the tissue of the target region.

In the examples of FIGS. 1 and 2, the source system comprises six laser diodes 23, 24, 25, 26, 27, 28 to generate five laser treatment beams and a detection laser beam, each of the beams being able to have its own wavelength and its own light power. The source system may also comprise, for example, three diodes that emit at 976 nm and three diodes that emit at 793 nm. Each of the diodes is associated with its own power supply unit 13, 14, 15, 16, 17, 18 and can be controlled individually by an electronic control unit 12.

According to another embodiment not shown, the system may comprise a plurality of monochromatic laser sources, for example, laser diodes, associated with each optical fiber. In this way, it is possible to select a given wavelength from a plurality of wavelengths for the laser beam intended to be transported and emitted by the optical fiber.

The central control unit 10 is connected to the electronic control unit 12 in order to transmit control signals to the control unit 12 to control the diodes individually and independently of each other. The central control unit 10 comprises a control unit for the laser beams 31 and a display unit 32. The beam control unit 31 is configured to send the control signals to the electronic control unit 12 in order to adjust the heat treatment parameters for each of the laser beams, which are the wavelength, the duration of the emission of the laser beam, the moment of emission of the laser beam, and the light power of the laser beam.

The control unit of the laser beams 31 also receives data coming from a unit for acquiring temperature measurements measured by a temperature sensor, for example, by the distal end of a detection fiber or by a thermocouple. The temperature measurement acquisition unit is housed in the source system 19. The display unit 32 makes it possible to display these temperature data coming from the temperature sensor.

The beam control unit 31 is configured to select the light power of the laser beam transmitted to each of the emitters. In the case where there are, for example, two laser beams generated for each optical fiber, it is possible to select, for example, one of the two light powers for each optical fiber.

The laser-beam control unit 31 is configured to select the wavelength of the light beam directed and emitted by each of the emitters in order to be able to modulate the penetration depth of the laser beam into the tissue of the target region. In the case where there are, for example, two laser beams generated for each optical fiber, it is possible to select, for example, one of the two wavelengths for each optical fiber.

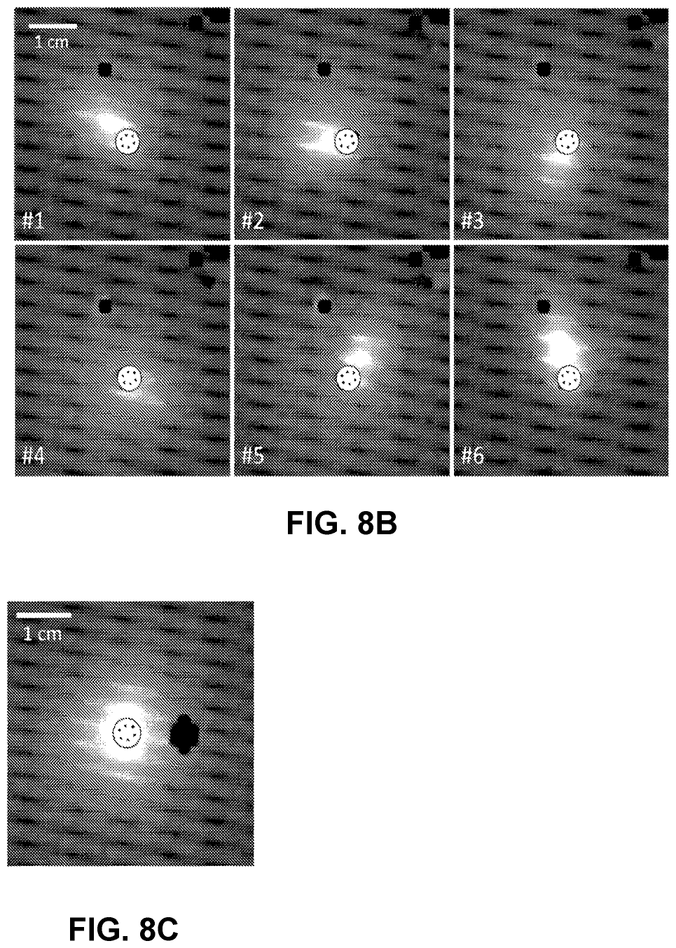

The beam control unit 31 is configured to select the emission duration of each treatment fiber and the emission moment of each treatment fiber so as to generate a thermal distribution with a specific geometric shape, adapted, in particular, to the geometric shape of the target region to induce a temperature variation. According to one embodiment, it is possible to activate the treatment optical fibers sequentially or simultaneously for part of the treatment optical fibers. Possible examples of use are illustrated in FIGS. 8A-8C, FIGS. 11-14.

Thus, in the case of the sheath of FIG. 4, each of the three treatment optical fibers directs and emits a light beam having its own light power P1, P2, P3, its own wavelength λ1, λ2, λ3, and its own emission duration t1, t2, t3. It is also possible to consider three different emission moments and three different emission times for the three treatment optical fibers.

The optical fibers are adapted to each transport a laser beam from the proximal end of the sheath to the distal end of the sheath. For this, the proximal end of the optical fibers of the sheath is connected to the system of laser sources 19 by connections provided for this purpose in the proximal area of the sheath, which will be detailed below.

FIG. 1 schematically shows an example of connection connectors intended to connect the optical fibers and the system of laser sources 19. In FIG. 1, the connection connectors are shown detached from each other. The sheath 150 comprises at its proximal end a single connector 130 intended to engage in optical connector 30 of the system of laser sources 19.

Advantageously, the light beams generated by the system of laser sources 19 are guided by a plurality of optical transmission fibers 43, 44, 45, 46, 47, 48 toward the optical connector 30. The optical transmission fibers are fibers equivalent to the optical fibers in the sheath, and may be of the same structure. The use of these optical transmission fibers makes it possible to install the system of laser sources 19 and the main control unit 10 in a room far from the room containing the MRI imaging device. Once the sheath is positioned in the body of the patient by the practitioner, the practitioner can use the central control unit 10 to adjust the various parameters of the laser beam during the treatment phase.

The use of the transmission fibers makes it possible to convey the light beams from the control part to a distance close to the patient, thus making it possible to use the device with multi-emitters of laser energy of the present disclosure together with an MRI device without creating interference between the MRI device and the electronic components of the system of laser sources 19. According to one embodiment, the length of the optical transmission fibers is between 10 meters and 15 meters. The optical transmission fibers are protected by a plastic sheath in order to protect them against any possible external disturbance.

According to one embodiment not shown, the connector 130 of the sheath 150 comprises connection tabs on a planar connection surface. The connector 30 of the source system comprises connection orifices on a connection surface. The connection tabs are able to be inserted into the orifices in order to engage the two connection connectors. Furthermore, when the connection tabs are inserted into the orifices, the two connection surfaces are in contact so that the ends of the optical fibers of the sheath come into contact, respectively, with the ends of the optical transmission fibers to connect the optical fibers together. The optical connectors are able to be mutually engaged to optically couple the plurality of optical fibers of the sheath to the plurality of optical transmission fibers of the system of laser sources 19.

The sheath 150 is thus detachably connected to the system of laser sources 19 via the optical connectors 30, 130, which allow easy manual connection and disconnection. Preferably, the connectors are made so as to be compatible with MRI.

FIG. 2 shows another example of optical connection between the optical sheath 150 and the source system 19. The optical treatment and detection fibers are each provided at their proximal end with an individual optical connector 133, 134, 135, 136, 137, 138, and the optical transmission fibers are also provided at their distal end with an individual optical connector 33, 34, 35, 36, 37, 38. The connectors allow easy manual connection and disconnection between the optical sheath and the source system 19.

The device with multi-emitters of laser energy of the present disclosure can be integrated into a heat treatment assembly.

According to one embodiment of the present disclosure and with reference to FIG. 7, such a heat treatment assembly comprises:
a device with multi-emitters as shown in FIGS. 1 and 2,
an MRI imaging device 50 configured to provide 3D anatomical images of the target region as well as temperature images of the target region throughout the duration of the treatment.

The assembly also comprises an image building unit 51 configured to provide 3D anatomical images and 3D temperature images from the data acquired by the MRI device. According to one embodiment, the central control unit 10 of the device with multi-emitters and the image building unit 51 can be integrated into a single entity.

The display unit 32 of the central control unit 10 is connected to the image building unit 51 and also makes it possible to display the temperature images in real time during the treatment and the temperature measurements transmitted by the temperature sensor of the device 1 with multi-emitters. The display unit 32 comprises a data input interface, thus enabling the practitioner to input the data to adjust the wavelength, the light power, the emission duration and the transmission moment of each of the light beams generated by the system of laser sources and intended to be transported and emitted by the optical fibers.

The target region is a region wherein the biological tissue is to undergo a temperature variation. This region must have a size suitable for ensuring the destruction of the entire pathological tissue while preserving tissues in the vicinity of the target region. The evaluation of the spatial extension of the target region is carried out by the practitioner in a so-called “pre-operative planning phase” from data relating to anatomical images of the target region. This phase also makes it possible to determine the complex geometric shape and the location of the target region.

As an example, FIG. 7 schematically shows a member comprising a target region, called Rc, surrounded by healthy regions.

During the pre-operative planning phase, from the anatomical image of the target region, the practitioner defines an intervention strategy that consists of defining:
a position of the distal end of the optical sheath relative to the target region;
the angular sector covered by each of the emitters;
the distal position of each of the emitters;
a wavelength for each of the treatment laser beams;
a light power for each of the treatment laser beams;
a duration of the deposition of laser energy in the target region for each of the emitters, this duration being able to be different for each emitter;
potentially different emission times for each of the emitters.

During the heat treatment phase, the practitioner can individually adjust the light power, the wavelength, the emission time and the moment of emission of the beams emitted by the optical emitters or fibers as a function of the temperature images transmitted by the MRI imaging device 50.

With reference to FIGS. 8A, 8B and 8C, two possible examples of use of the same optical sheath are described. FIG. 8A schematically shows an optical sheath 150 with a radial distribution of six optical fibers positioned at the distal end of the sheath. Each of the optical fibers is able to emit a laser beam digitally referenced F1, F2, F3, F4, F5 and F6. FIG. 8B shows six temperature images obtained by the MRI imaging device. Each temperature image is obtained simultaneously with the activation of a single optical fiber, each optical fiber being sequentially activated one after the other. The temperature images clearly show six heated zones in distinct angular sectors. Furthermore, the temperature distributions around the optical sheath are more or less extended, due to a different power delivered over each optical fiber. FIG. 8C shows a temperature image obtained during the simultaneous activation of the six optical fibers with the same power. The temperature image shows a substantially circular rise in temperature around the optical sheath 150.

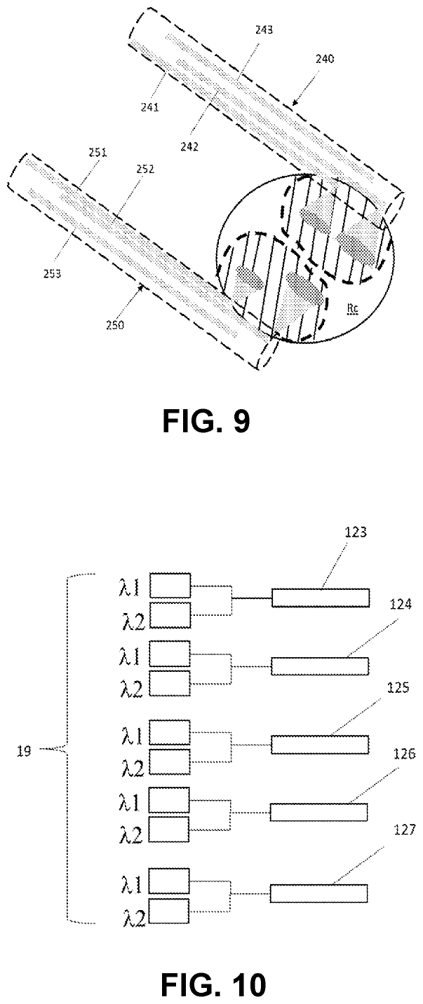

Referring to FIG. 9, an example of possible use of two sheaths 240, 250 whose distal ends are positioned on either side of a target region Rc. Each of the sheaths 240, 250 here, respectively, comprises three optical fibers 241, 242, 243, 251, 252, 253. By virtue of the technical solution of the present disclosure, it is thus possible to activate only the two optical fibers 241, 242, 251, 252 whose laser beams are emitted in the direction of the target region, and to leave the third optical fiber inactive for which the emitted laser beam covers an angular sector located in a healthy region to be preserved of the biological tissue. Furthermore, it is also possible to select a different wavelength, light power, and laser energy deposition duration for each of the four laser beams emitted so as to be able to generate a 3D thermal distribution that corresponds to the 3D geometric shape of the target region.

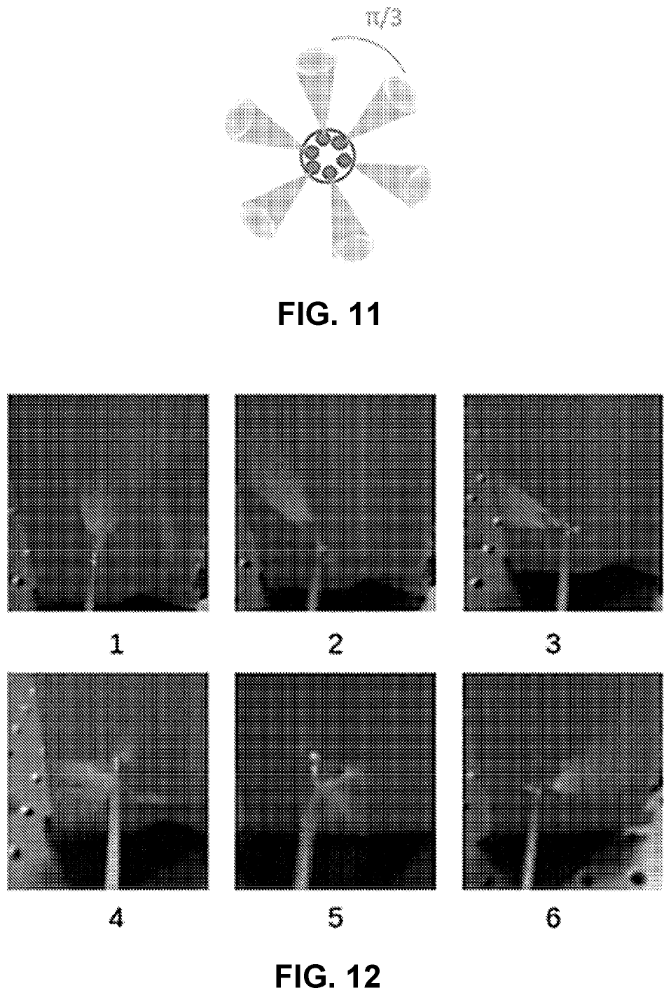

Referring to FIGS. 11-14, an example use of a laser device 1 with multi-emitters of laser energy comprising six optical fibers for heat-treating a target region of a biological tissue is described below.

With reference to FIG. 11, the laser device comprises six optical fibers, encapsulated in a single sheath. The diameter of each optical fiber is 200 μm and the final diameter of the sheath is 2 mm. The distal end of each fiber was machined to ensure radial propagation of each individual laser beam in a different direction, in order to be able to achieve an angular coverage of 60° for each fiber, the six fibers being distributed along a radial symmetry over 360°. In the context of the example presented, each laser fiber is connected to a laser diode having a wavelength of 976 nm and a maximum power of 9 W. Each laser diode is commanded individually by a laser-beam control unit configured to control the laser diodes so as to dynamically adjust the wavelength, the light power, the duration of the laser energy deposition and the moment of emission of each of the guided laser beams emitted by the optical fibers toward the target region during firing. This adjustment is possible at the start of the transmission by the control unit and adjustable during transmission.

FIG. 12 shows six photographs of the individual emission beams of each optical fiber of the device of FIG. 11. Each individual laser beam is visualized by connecting a laser diode in the visible domain, for example, at a wavelength of 532 nm.

The six photographs qualitatively show that each optical fiber clearly illuminates a different angular sector, with light beams whose characteristics appear slightly different as a function of the emitting channels.

The operation of the laser device of FIG. 11 is then validated by MRI thermometry. The distal end of the fibers surrounding the sheath, referred to herein as a probe, is introduced into a gel containing gelatin. The assembly is positioned at the center of a magnetic resonance imaging (MRI) apparatus operating at 1.5 T. Scout imaging is carried out in order to view the probe and the gel, and to position the thermometry cuts perpendicular to the axis of the probe by encompassing the zone illuminated by all of the fibers. MRI temperature imaging is carried out with a fast echo planar imaging sequence and comprises 10 cuts (resolution in the plane equal to 1.4 mm, cutting thickness equal to 3 mm) that are continuously recorded (dynamic imaging) over a total duration of several minutes with a refresh rate of 2 seconds. In the following examples, the parameters of the thermometry acquisition technique are: echo time of 18 ms, field of view of 180×180 mm2, tilt angle of 60°, GRAPPA acceleration by a factor of 2, bandwidth per pixel of 1446 Hz. The images are processed in real time by a computing unit to obtain the temperature maps from phase images. These temperature maps are displayed color-coded in real-time in a graphic interface. Characteristic points can be identified in the image to select one or more of the pixels wherein the temporal evolution of the temperature will also be displayed.

In a first example of use, each diode is supplied sequentially at a power of 4.2 W for 30 seconds, with a pause of 10 seconds between the emission of each diode.

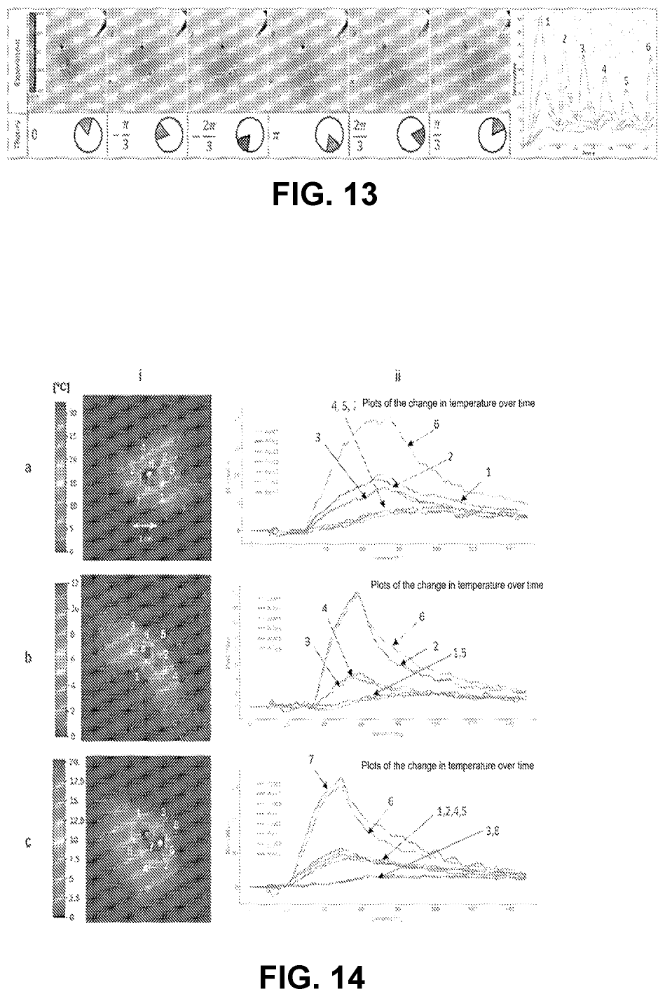

FIG. 13 shows six temperature images obtained by MRI during sequential activation of each emitting channel, at the moment corresponding to when the emission of each of the six optical fibers stopped (maximum temperature rise). For each image, the angular sector covered by the fiber is indicated, for the activated diode. The graph on the right shows the change in temperature in six pixels referenced from 1 to 6, each pixel being selected in an angular sector covered by a different fiber.

The results show a rise in temperature in different angular sectors for each fiber, in agreement with FIG. 12 (qualitative characterization of the laser emission). The rise in temperature is less pronounced for fiber number 5 (angular sector 2π/3 in FIG. 13), which is consistent with the photo of the illumination produced by this fiber in FIG. 12. The graph on the right shows the change in six different pixels positioned in each of the six angular sectors covered by each of the fibers. A sequential rise of the temperature is observed as a function of the activated laser diode.

FIG. 14 shows three results obtained by differently activating the laser diodes supplying each of the optical fibers, in order to generate a thermal distribution in the shape of a triangle, an ellipse or a semicircle.

FIG. 14 shows three temperature images (a), (b) and (c) obtained by MRI thermometry for three different activation configurations. The variation in temperature in the heated region is represented by a variation in gray level displayed on the left of the images. Configuration (a) corresponds to simultaneous activation of three fibers 2, 4 and 6, with a power of 2 W and for a duration of 60 seconds. The temperature image has a triangular shape. Configuration (b) corresponds to simultaneous activation of two fibers 3 and 6, with a power of 1.5 W and for a duration of 25 seconds. The temperature image has an ellipse shape. Configuration (c) corresponds to simultaneous activation of four fibers 1, 2, 3 and 6, with a power of 1.5 W and for a duration of 30 seconds. The temperature image has a semicircle shape.

Using a plurality of laser energy emitters, each covering a different angular sector and at different positions along the sheath, makes it possible to generate an adjustable 3D temperature distribution relative to the arbitrary geometric shape of the target region. Due to this flexibility as to the geometry of the thermal lesion created, the present disclosure is particularly suitable for treating cardiac fibrillations, for treating tumors of various organs, such as the abdomen and pathological brain regions.

Furthermore, the control is more precise in terms of depth of penetration of the light beam into the tissue of the target region by modulating the wavelength of the beams emitted by each of the emitters.

Finally, when the device with multi-emitters of laser energy is coupled with an MRI imaging device in a heat treatment assembly, it is possible to modulate the light power, emission duration, and moment of transmission of each of the emitters in order to adjust the deposition of laser energy over time and in space from the temperature images obtained by the MRI imaging device.

The present disclosure is not limited to the embodiments described above by way of non-limiting example. It encompasses all alternative embodiments that can be envisaged by a person skilled in the art. It should be understood, in particular, that logical changes can be made. In addition, the detailed description of embodiments of the present disclosure should not be interpreted as limiting the order of the steps and sub-steps.

1. A laser device with multi-emitters of laser energy for heat-treating a target region of a biological tissue, the laser device comprising:
at least one sheath having a longitudinal axis and comprising a proximal end and a distal end intended to be placed facing the target region;
at least two optical fibers extending, in the sheath, between the proximal end and the distal end, each of the optical fibers being suitable for guiding a heat-treatment laser beam to the target region and depositing laser energy in the target region, the distal ends of at least two optical fibers being configured so that each emits a laser beam in a different emission direction with respect to the longitudinal axis of the sheath;
a system of laser sources configured to generate at least two laser beams, the at least two laser beams having different or identical wavelengths with an adjustable light power; and
a laser-beam control unit configured to control the system of laser sources so as to select the wavelength, the light power, the duration of the deposition of laser energy and the moment of emission of each of the laser beams guided and emitted by the optical fibers in the direction of the target region so as to dynamically generate and adjust a 3D thermal distribution having a geometric shape matching the geometric shape of the target region.

2. The device according to claim 1, wherein the distal end of at least two optical fibers is positioned at a different distance from a surface of the distal end of the sheath.

3. The device according to claim 1, wherein the distal end of the optical fibers is configured to emit a laser beam in an emission direction oriented at an angle α between 0 and 180° with respect to the longitudinal axis of the sheath.

4. The device according to claim 1, wherein the system of laser sources comprises a plurality of monochromatic laser sources.

5. The device according to claim 1, wherein the system of laser sources is adapted to generate at least two laser beams of different laser wavelength for each optical fiber.

6. The device according to claim 1, further comprising a plurality of transmission optical fibers configured to transmit the laser beams generated by the system of laser sources to the optical fibers of the sheath.

7. The device according to claim 1, further comprising a temperature sensor.

8. The device according to claim 7, wherein the temperature sensor is a detection optical fiber for detecting a temperature variation in the target region.

9. The device according to claim 8, wherein the plurality of heat treatment optical fibers is distributed along a radial symmetry around the detection optical fiber.

10. The device according to claim 6, further comprising connector configured to connect the optical fibers of the sheath with the optical transmission fibers of the system of laser sources.

11. The device according to claim 1, wherein the sheath comprises at least one lumen adapted to inject a therapeutic substance under pressure toward the target region.

12. The device according to claim 1, wherein the sheath comprises a closed cooling circuit adapted for transporting a cooling liquid for cooling a part of the distal end of the sheath.

13. The device according to claim 12, wherein the closed cooling circuit comprises at least two lumens provided in the sheath.

14. The device according to claim 12, wherein the closed cooling circuit is formed by a cooling sheath surrounding the sheath comprising the optical fibers and a lumen provided in the sheath.

15. An assembly for heat treatment of a target region of a biological tissue, the assembly comprising:
a laser device with multi-emitters of laser energy according to claim 1 for treating the target region; and
a magnetic resonance imaging device configured to generate anatomical images and thermometric images of the target region.

16. The device according to claim 2, wherein the distal end of the optical fibers is configured to emit a laser beam in an emission direction oriented at an angle α between 0 and 180° with respect to the longitudinal axis of the sheath.

17. The device according to claim 16, wherein the system of laser sources comprises a plurality of monochromatic laser sources.

18. The device according to claim 17, wherein the system of laser sources is adapted to generate at least two laser beams of different laser wavelength for each optical fiber.

19. The device according to claim 18, further comprising a plurality of transmission optical fibers configured to transmit the laser beams generated by the system of laser sources to the optical fibers of the sheath.

20. The device according to claim 19, further comprising a temperature sensor.
